(19) United States
(12) Patent Application Publication
(10) Pub. No.: US 2023/0001119 A1
(43) Pub. Date: Jan. 5, 2023

(54) SYSTEMS AND METHODS FOR INCREASING NITROGEN MONOXIDE CONCENTRATION AND REMOVING NITROGEN DIOXIDE FROM A GAS STREAM

(71) Applicant: Third Pole, Inc., Waltham, MA (US)

(72) Inventors: Robert G. Richardson, Shingletown, CA (US); Gregory W. Hall, Belmont, MA (US)

(73) Assignee: Third Pole, Inc., Waltham, MA (US)

(21) Appl. No.: 17/773,369

(22) PCT Filed: Oct. 30, 2020
(86) PCT No.: PCT/US2020/058390

§ 371 Date: Apr. 29, 2022

Related U.S. Application Data
(60) Provisional application No. 62/929,338, filed on Nov. 1, 2019.

(51) Int. Cl. A61M 16/00; A61M 16/06; A61L 29/14; A61L 29/10; A61K 33/00

(52) U.S. Cl. CPC A61M 16/0093; A61M 16/0666; A61L 29/14; A61L 29/106; A61K 33/00; A61M 2202/0275

(57) ABSTRACT

The presently disclosed embodiments relate to devices and methods for efficiently removing the nitrogen dioxide (NO₂) from a gas stream while enhancing the concentration of nitric oxide (NO) in the gas stream without making any reaction byproduct that will adversely influence a respiratory treatment. The devices and methods for nitric oxide (NO) generation and nitrogen dioxide (NO₂) removal or scrubbing can be embedded into other therapeutic devices or used alone.

### RELATED APPLICATIONS

This application claims priority to and the benefit of U.S. Provisional Application No. 62/929,338, filed Nov. 1, 2019, the disclosure of which is hereby incorporated by reference in its entirety for all purposes.

### FIELD

Aspects of the presently disclosed embodiments relate to compositions, systems, devices and methods for efficiently removing the nitrogen dioxide (NO₂) from a gas stream while enhancing the concentration of nitric oxide (NO) in the gas stream without exposing a patient to any reaction byproduct that will adversely influence a respiratory treatment of the patient.

### BACKGROUND

Nitric oxide has found to be useful for treatment of disease, particularly cardiac and respiratory ailments. Medical research has demonstrated that specific respiratory complications are effectively treated by the regulated exposure of pulmonary tissue to NO. An effective and non-toxic treatment requires the removal of NO₂ from the gas stream.

### SUMMARY

Aspects of the disclosure are directed to a device for delivering a gas containing nitric oxide (NO) and/or nitrogen dioxide (NO₂). In some embodiments, the device comprises one or more inlets; one or more lumens comprising one or more surfaces configured to be in contact with the NO/NO₂ containing gas stream, wherein the one or more gas contact surfaces comprise a nitrogen dioxide sequestering (NO₂-sequestering) molecule; and one or more outlets. In some embodiments, the device is a nasal cannula.

In some embodiments, the NO₂-sequestering molecule is molecule that sequesters NO₂. In some embodiments, the NO₂-sequestering molecule can be reset. In some embodiments, the NO₂-sequestering molecules include TEMPO, 4-substituted TEMPO derivatives, PROXYL and derivatives, TEMPA and derivative, calcium hydroxide, and sodium hydroxide or combinations thereof.

In some embodiments, the one or more lumens are a component of a multilayer and/or multilumen structure. In some embodiments, the one or more lumens are textured.

In some embodiments, the device comprises one or more gas contact surfaces that contain an indicator indicative of the propensity of the NO₂-sequestering to sequester NO₂. In some embodiments, the device comprises one or more gas contact surfaces comprise the NO₂-sequestering molecules dissolved therewithin. In some embodiments, the device comprises one or more gas contact surfaces that comprise the NO₂-sequestering molecules dispersed therewithin. In some embodiments, the device comprises one or more gas contact surfaces that comprise the NO₂-sequestering molecules chemically bound thereto.

Aspects of the disclosure are directed to a device for delivering gas containing nitric oxide (NO) and/or nitrogen dioxide (NO₂) comprising one or more inlets; one or more lumens comprising one or more surfaces configured to be in contact with the NO/NO₂ containing gas stream, wherein the one or more gas contact surfaces comprise a nitrogen dioxide sequestering (NO₂-sequestering) molecule; an electrically conductive material configured to be in contact with the nitrogen dioxide NO₂-sequestering molecule; and one or more outlets. In some embodiments, the device is a nasal cannula.

In some embodiments, the one or more lumens are a component of a multilayer and/or multilumen structure. In some embodiments, the one or more lumens are textured.

In some embodiments, the device comprises one or more gas contact surfaces that contain an indicator indicative of the propensity of the NO₂-sequestering to sequester NO₂. In some embodiments, the device comprises one or more gas contact surfaces comprise the NO₂-sequestering molecules dissolved therewithin. In some embodiments, the device comprises one or more gas contact surfaces that comprise the NO₂-sequestering molecules dispersed therewithin. In some embodiments, the device comprises one or more gas contact surfaces that comprise the NO₂-sequestering molecules chemically bound thereto.

In some embodiments, the NO₂ sequestering molecule is a molecule that sequesters NO₂. In some embodiments, the NO₂ sequestering molecule includes TEMPO, 4-substituted TEMPO derivatives, PROXYL and derivatives, TEMPA and derivative, calcium hydroxide, sodium hydroxide or combinations thereof.

Aspects of the disclosure are directed to a device for delivering nitric oxide (NO), the device comprising one or more lumens, the lumen having one or more surfaces containing a nitrogen dioxide (NO₂) sequestering molecule, wherein the one or more surfaces are configured to contact a gas stream; one or more inlets configured to receive the gas stream comprising NO, NO₂ or NO and NO₂; and one or more outlets configured to deliver NO enriched gas.

In some embodiments, the NO₂ sequestering molecule comprises one or more nitroxide radical containing compounds. In some embodiments, the NO₂-sequestering molecule is a molecule that sequesters NO₂. In some embodiments, the NO₂-sequestering molecule is a molecule that reacts with NO_{2.} In some embodiments, the NO₂-sequestering molecule is a molecule that selectively binds with NO₂. In some embodiments, the NO₂-sequestering molecule includes TEMPO, TEMPA, TEMPO derivatives, TEMPO analogs or combinations thereof.

In some embodiments, the device comprises one or more gas contact surfaces that comprise an electrically conductive polymer. In some embodiments, the NO₂-sequestering molecule is embedded into the electrically conductive polymer. In some embodiments, the NO₂-sequestering molecule is attached to the electrically conductive monomer or polymer. In some embodiments, the NO₂-sequestering molecule is embedded in the electrically conductive monomer/polymer, wherein the electrically conductive monomer/polymer conveys variations in electrical bias to the NO₂-sequestering molecule, thereby regulating the chemical reactivity of the NO₂-sequestering molecule. In some embodiments, the NO₂-sequestering molecule is chemically attached directly or through a linker molecule to the electrically conductive monomer that is part of the polymer.

In some embodiments, the NO₂-sequestering molecule includes TEMPO, TEMPA, TEMPO derivatives, TEMPO analogs or combinations thereof and wherein the one or more surfaces comprising the TEMPO, TEMPA, TEMPO derivatives, TEMPO analogs or combinations thereof are configured to be reset.

In some embodiments, the one or more surfaces comprise the TEMPO, TEMPA, TEMPO derivatives, TEMPO analogs or combinations thereof and are configured to be reset upon application of an electrical potential to the one or more surfaces so that NO₂ is released from the one or more surface.

In some embodiments, the NO₂-sequestering molecule includes TEMPO, TEMPA, TEMPO derivatives, TEMPO analogs or combinations thereof and the TEMPO, TEMPA, TEMPO derivatives, TEMPO analogs or combinations thereof are bonded to electrically-conductive monomer that is part of the polymer so that changes in electrical potential applied to the monomer/polymer increase or decrease the affinity of the TEMPO, TEMPA, TEMPO derivatives, TEMPO analogs, or combinations thereof, for NO₂.

In some embodiments, the TEMPO, TEMPA, TEMPO derivatives, TEMPO analogs, or combinations thereof, are covalently bonded to electrically-conductive monomer.

In some embodiments, the one or more lumens are textured.

In some embodiments, the device is a nasal cannula. In some embodiments, the device further comprises a coating on one or more inspiratory-gas-contacting surfaces within the nasal cannula.

In some embodiments, the device further comprises an electrochromic gel adjacent or integrated into the conductive polymer and configured to change color based on an changes in electrical bias/electrical voltage applied to the conductive polymer.

In some embodiments, the device further comprises an indicator indicative of the propensity of the TEMPO, TEMPA, TEMPO derivatives, TEMPO analogs, or combinations thereof, to sequester NO₂.

Aspects of the disclosure relate to a method for enriching a gas stream in nitric oxide, the method comprising delivering a gas stream comprising nitric oxide (NO), nitrogen dioxide (NO₂) or NO and NO₂ onto a substrate comprising a NO₂-sequestering molecule, such that the gas stream contacts the NO₂-sequestering molecule; and enriching the gas stream with NO by the removing NO₂ from the gas stream thereby generating a NO enriched gas stream.

In some embodiments, the method further comprises storing the NO enriched gas stream. In some embodiments, the method further comprises delivering the NO enriched gas stream.

In some embodiments, the NO₂-sequestering molecule selectively adsorbs NO₂. In some embodiments, the NO₂-sequestering molecule selectively reacts with NO₂. In some embodiments, the NO₂-sequestering molecule is one of TEMPO, TEMPO derivatives, TEMPO analogs or combinations thereof.

In some embodiments, the substrate comprises an electrically conductive monomer that is part of an electrically conductive polymer. In some embodiments, the NO₂-sequestering molecule is attached to the substrate. In some embodiments, the NO₂-sequestering molecule is covalently bonded to the electrically conductive monomer that is part of a polymer. In some embodiments, the NO₂-sequestering molecule is embedded into the electrically conductive polymer.

In some embodiments, the method further comprises altering the TEMPO molecules, TEMPO derivatives, TEMPO analogs or combinations thereof by applying an electrical charge to the TEMPO molecules, TEMPO derivatives, TEMPO analogs or combinations thereof.

In some embodiments, the substrate comprises an electrochromic gel that is attached to or included in the electrically conductive polymer, wherein the electrochromic gel change color as a result of a change in an electrical charge being applied to the electrically conductive polymer, thereby indicating the propensity of the TEMPO, TEMPO derivatives, TEMPO analogs, or combinations thereof, to react with NO₂.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 is a diagram of generic nitronyl nitroxide radicals.

FIG. 2 shows examples of conjugated polymers according to the presently disclosed embodiments.

FIG. 3 shows examples of high surface area monomer and polymer extrusions according to the presently disclosed embodiments.

FIG. 4 shows an example of a color-changing gel located between TEMPO-containing sleeve and a structural conduit according to the presently disclosed embodiments.

FIG. 5 shows exemplary molecules within the TEMPO family that can be used for selective NO₂-absorption according to the presently disclosed embodiments. FIG. 5 shows TEMPO; TEMPO as free radical; 4-Amino-TEMPO, free radical; 4-Oxy-TEMPO-d16, free radical (e.g. for tracing reactions); 4-Acetamido-TEMPO, free radical; 4-Oxo-TEMPO, as free radical; 4-Hydroxy-TEMPO, free radical; 4-Hydroxy-TEMPO benzoate, free radical; 4-Carboxy-TEMPO, free radical; and TEMPA.

FIG. 6 shows exemplary molecules within the PROXYL family that can be used for selective NO₂-adsorption according to the presently disclosed embodiments.

FIG. 7A and FIG. 7B shows different examples of tubes.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

As used herein, “NO₂-sequestering” and “NO₂ reactive” may be used interchangeably. Compositions, methods, and devices comprising “NO₂ sequestering” and “NO₂ reactive” molecules are provided for the active reduction of NO₂ from the gas stream through reversible chemical removal, physical removal or inactivation of NO₂ or irreversible chemical removal, physical removal or inactivation of NO₂. In some embodiments, using such composition, methods and devices the patient is exposed to clinically relevant lower concentrations of NO₂. Clinically relevant levels can be on the order of 0.05 ppm to 25 ppm, depending on the length of exposure. Clinical relevance of a NO₂ concentration depends on the duration of exposure. In some embodiments involving brief exposure (8 hours or less), NO₂ levels are kept below 5 ppm. In other embodiments involving prolonged exposure, NO₂ levels are kept below 0.1 ppm.

Medical research has demonstrated that specific respiratory complications are effectively treated by the regulated exposure of pulmonary tissue to NO. An effective and non-toxic treatment requires the removal of NO₂ from the gas stream. Aspects of the presently disclosed embodiments relate to chemical compositions, devices and methods for efficiently removing the NO₂ from a gas stream while enhancing the concentration of NO in the gas stream without making any reaction byproduct that will adversely influence the respiratory treatment. In some embodiments, the compositions and methods are provided at ambient temperatures. The compositions and methods described herein are applicable to, but not limited to, NO generation devices, NO delivery devices and inspiratory gas conduits, tubing and circuits that transfer NO-containing gas to a patient such as nasal cannula, ventilator tubing, ET Tubes, humidifier chambers, oxygen generators, patient monitors, and similar devices.

Aspects of the methods, devices and compositions described herein overcome the propensity of NO₂ generation in a gas stream at any point up to the point where the gas is administered to a subject in need thereof. In some embodiments, the subject in need thereof is a human. In some embodiments, the composition or device comprises minerals, small molecules or polymers and combinations thereof that are dissolved, dispersed, or suspended and combinations thereof to effect sufficient surface areas containing nitroxide radicals that are exposed to the gas stream. Nitroxide radicals are also known as nitroxides and aminoxyl radicals. By any of the three names, the chemical species is characterized by containing the R₂N—O* functional group. In some embodiments, the methods for NO₂ scrubbing from a gas stream occurs at points where stable nitroxide radicals embedded or attached to monomers or polymers that contain high surface areas are exposed to the gas stream.

In some embodiments, the integration of nitroxide radical compounds with polymers or monomers provides a flexible material with high surface area and covalently bonded stable nitroxide radical compound surface area that prevents the formation of dust even when the material is flexed.

Aspects of the presently disclosed embodiments relate to methods for regulating the chemical reactivity of the stable nitroxide radical compounds by applying an electrical potential to an electrically conductive polymer described herein used as the substrate composed of monomers to which the stable nitroxide radical compounds are covalently bonded or embedded.

Aspects of the presently disclosed embodiment relate to methods for regulating the chemical reactivity of the stable nitroxide radical compounds by applying an electrical potential to and electrically conductive polymer described herein that is bonded or attached to a non-conductive polymer that is composed of monomers to which the stable nitroxide radical compounds are covalently bonded or embedded.

Aspects of the presently disclosed embodiments relate to a method for delivering nitric oxide (NO), the method comprising delivering a gas stream comprising nitric oxide and nitrogen dioxide (NO₂) onto a substrate comprising a NO₂ sequestering molecule, such that the NO₂ in the gas stream contacts the NO₂ sequestering molecule and is captured or modified into nitrogen monoxide or other compounds, wherein the NO₂ sequestering molecule is one of TEMPO (2,2,6,6-Tetramethyl-1-piperidinyloxy, free radical), and 4 substituted TEMPO, TEMPO derivatives (4 hydroxy TEMPO, 4 amino TEMPO, 4 oxy TEMPO), TEMPO analogs or combinations and variations thereof; enriching the gas stream with NO and removing nitrogen dioxide NO₂ from the gas stream, and delivering the NO enriched gas stream.

In some embodiments, the NO₂-sequestering molecule selectively adsorbs NO₂. In some embodiments, the NO₂-sequestering molecule selectively reacts with the NO₂. In some embodiments, the NO₂-sequestering molecule can be in a crystalline form of any particle size. In some embodiments, smaller particle sizes can provide greater surface area, thereby increasing NO₂ absorption or reaction for a given volume or surface area.

In some embodiments, the substrate is not electrically conductive but can be influenced (electronically biased) by electrical current in an adjacent conducting polymer or other material. In some embodiments, the substrate comprises an electrically conductive material. In some embodiments, the NO₂-sequestering molecule is a covalently bonded component of the substrate. In some embodiments, the NO₂-sequestering molecule is embedded into the substrate. In some embodiments, the NO₂-sequestering molecule is attached to the substrate. The substrate material may contain or be comprised of one or more monomers, polymers, or additives.

In some embodiments, the NO₂-sequestering molecule is a member of the group of TEMPO molecules, TEMPO derivatives, TEMPO analogs or combinations thereof. The NO₂-sequestering molecules can further be affected by applying an electrical charge to the TEMPO molecules, TEMPO derivatives, TEMPO analogs or combinations thereof. In some embodiments, the substrate includes an electrochromic gel on or in an electrically conductive polymer, wherein a change in the color of the electrochromic gel is indicative of the electrical bias on the polymer or monomer and that influences or indicates the propensity of the TEMPO, TEMPO derivatives, TEMPO analogs or combinations thereof to interact with NO₂.

Aspects of the presently disclosed embodiments relate to a device for delivering nitric oxide (NO), the device comprising a nasal cannula including one or more intra-lumen, the intra-lumen having a surface comprising a NO₂-sequestering material, and wherein the intra-lumen surface is configured to contact a gas stream; an inlet to receive the gas stream comprising NO, NO₂ or NO and NO₂; and an outlet configured to deliver NO enriched gas.

It should be appreciated that depending on the efficiency of sequestration of NO₂ molecules, NO enriched gas existing the device may still have trace amounts of NO₂. 100% scrubbing is not essential so long as NO₂ levels are clinically acceptable, 0.1 to 5 ppm NO₂, depending on duration of exposure. In some embodiments, the NO treatment is sufficiently brief (a single breath to 5 minutes or less), that NO₂ levels up to 25 ppm are acceptable. In some embodiments, multiple scrubbers are used in series to sequester additional NO₂ molecules.

In some embodiments, the NO₂-sequestering molecule comprises TEMPO, TEMPO derivatives, TEMPO analogs or combinations thereof. In some embodiments, NO₂-sequestering molecule selectively adsorbs NO₂. In some embodiments, NO₂-sequestering molecule selectively reacts with NO₂. In some embodiments, the surface that is exposed to the gas stream comprises an electrically conductive polymer. In some embodiments, the surface that is exposed to the gas stream is a non-conducting polymer. In some embodiments, the NO₂ reactive molecule is embedded into the electrically conductive monomer that is part of a polymer. In some embodiments, the NO₂ reactive molecule is chemically attached to the electrically conductive monomer within a polymer. In some embodiments, the electrically conductive monomer or polymer is configured to alter the TEMPO molecules, TEMPO derivatives, TEMPO analogs, or combinations thereof upon application of an electrical charge to the TEMPO molecules. In some embodiments, the TEMPO molecules, TEMPO derivatives, TEMPO analogs, or combinations thereof can be chemically attached to the electrically conductive monomer within a polymer through another molecule that acts as a linker. In some embodiments, the surface comprising the TEMPO molecules, TEMPO derivatives, TEMPO analogs, or combinations thereof can be configured to be reset upon application of an electrical potential to the surface so that NO₂ is released from the TEMPO molecules, TEMPO derivatives, TEMPO analogs, or combinations thereof. In some embodiments, the TEMPO molecules, TEMPO derivatives, TEMPO analogs, or combinations thereof are covalently bonded to electrically-conductive monomer within a polymer so that changes in electrical potential applied to the monomer or polymer increase or decrease the TEMPO molecule (or TEMPO derivative or TEMPO analogs, or combinations thereof) affinity for NO₂.

In some embodiments, the device further comprises a coating on one or more inspiratory-gas-contacting surfaces within the nasal cannula or other device that exposes the gas to the NO₂-sequestering molecules.

In some embodiments, the device further comprises an electrochromic gel configured to change color based on an electrical voltage. In some embodiments, the color change of the electrochromic gel is indicative of the propensity of the TEMPO molecules, TEMPO derivatives, TEMPO analogs, or combinations thereof to bind with or remove NO₂.

In some embodiments, the methods and composition provided herein can also be used in NO gas-handling components for wound healing and blood-gas exchange (e.g. Extracorporeal membrane oxygenation (ECMO)).

The presently disclosed embodiments relate to devices and methods for nitric oxide (NO) generation and nitrogen dioxide (NO₂) scrubbing that can be embedded into other therapeutic devices or used in other applications. Some embodiment utilizes organic functional groups, and specifically organic radicals, with active sites for selective removal of gas phase nitrogen dioxide (NO₂) and simultaneous generation of gas phase nitrogen monoxide (NO) during a chemical reaction that involves solid and gas phase interface with high surface area compounds of the stable nitroxide radical groups.

Medical research has demonstrated that specific respiratory complications are effectively treated by the regulated exposure of pulmonary tissue to NO. An effective and non-toxic treatment requires the removal of NO₂ from the gas stream. NO₂ scrubbing is necessary because literature studies have shown that NO has the propensity to convert to NO₂ at ambient temperatures as demonstrated by the high Kc value in Equation 1 reports (Kimbrough, Sue “NO to NO₂ conversion, ATMOSPHERIC ENVIRONMENT. Elsevier Science Ltd, New York, N.Y., 165:23-24, (2017)).

2NO(g)+O₂(g)↔2NO₂(g); Kc=6.9×10⁵ [Equation 1]

Aspects of the presently disclosed embodiments overcome the short-comings of other chemical reactions that do not selectively remove NO₂ while generating NO. For example, nonionic chlorine dioxide (ClO₂) removes both NO and NO₂ from a gas stream and nitronyl nitroxides (FIG. 1) act as an oxidant in the conversion of NO to form NO₂ in a gas stream.

Aspects of the present disclosure are directed to systems, methods and devices for nitric oxide generation for use with various ventilation devices. Aspects of the presently disclosed embodiments provide compositions, methods and devices for efficiently removing the NO₂ from a gas stream while enhancing the concentration of NO in the gas stream at ambient temperatures without making any reaction byproduct that will adversely influence the respiratory treatment of a patient. It should be appreciated that depending on the efficiency of sequestration of NO₂ molecules, NO enriched gas exiting the device may still have trace amounts of NO₂. It should be appreciated that depending on the efficiency of sequestration of NO₂ molecules, NO enriched gas existing the device may still have trace amounts of NO₂. In some embodiments, multiple scrubbers are used in series to sequester additional NO₂ molecules. 100% scrubbing is not essential so long as NO₂ levels are clinically acceptable, depending on duration of exposure. For example, NO₂ levels can range from 0.1 to 5 ppm NO₂. In some embodiments, gas exiting the device contains zero to 10 ppb of NO₂. In other embodiments, gas existing the device contains 10 pppm NO₂. Whether or not a level of NO₂ exiting a device is acceptable depends on the NO dose, the exposure time, and presence/absence of additional scrubbing downstream.

Aspects of the presently disclosed embodiments provide polymeric devices comprising stable nitroxide radical compounds attached to monomeric compositions. Other aspects of the presently disclosed embodiments provide methods for regulating compounds that can have their chemical reactivity changed by applying an electrical potential to the conductive polymer used as the substrate for the stable nitroxide radical compounds.

According to some aspects of the presently disclosed embodiments, the compositions or devices comprise a selective NO₂-sequestering molecule. In some embodiments, the selective NO₂ sequestering molecules include, but are not limited to, TEMPO, TEMPO, analogs thereof or derivatives thereof. TEMPO, also known as (2,2,6,6-Tetramethylpiperidin-1-yl)oxyl or (2,2,6,6-tetramethylpiperidin-1-yl)oxidanyl, has been used as a versatile class of organic oxidants, both in the chemical bond producing and chemical bond encouraging (catalytic) modes.

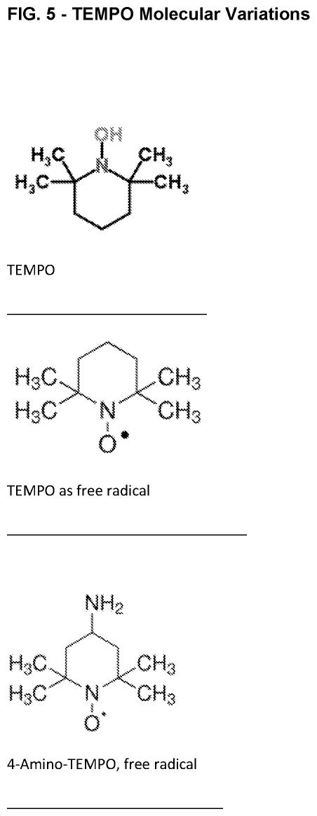

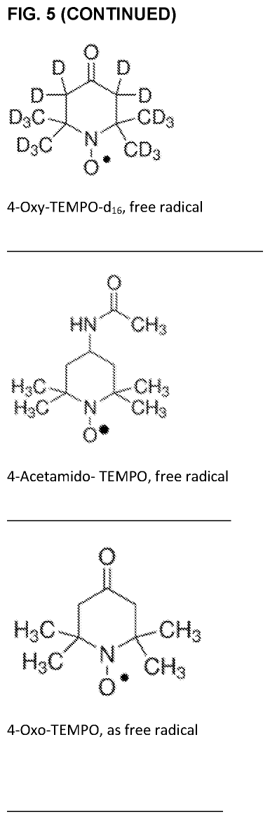

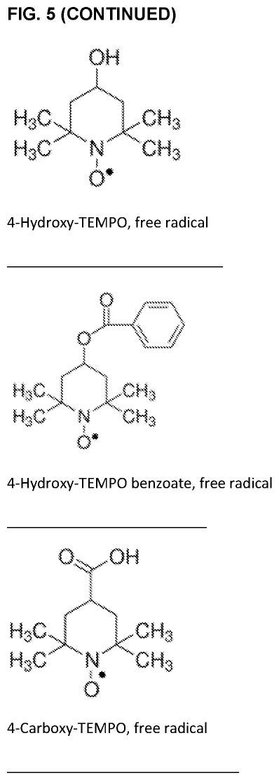

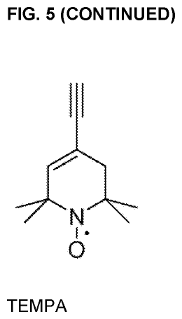

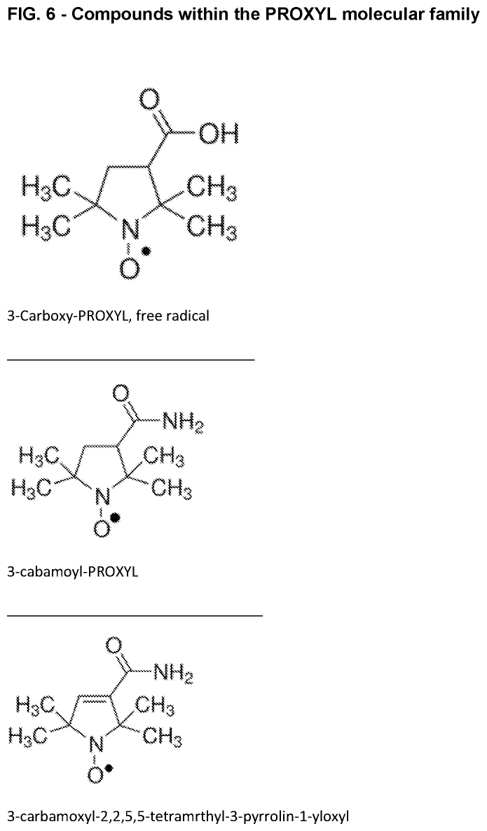

Provided herein are methods for implementing a selective NO₂ sequestering material into an NO-delivery device. In some embodiments, TEMPO is provided as a non-limiting example, and it should be understood that there are many types of molecules that can have selective NO₂-adsorption, as shown in FIG. 5 and FIG. 6, that can be deployed in a similar fashion.

It is this oxoammonium species that effectively reacts with NO₂ as shown in Equation 2. It is important to note that the reactions shown in Equation 2 shows that one mole of TEMPO reacts with and removes two moles of NO₂ from the gas stream while creating one mole of NO. This reaction occurs rapidly and with high efficiency. It is also noteworthy that in this series of reactions the TEMPO does not react with NO in the gas stream but on the contrary, generates NO.

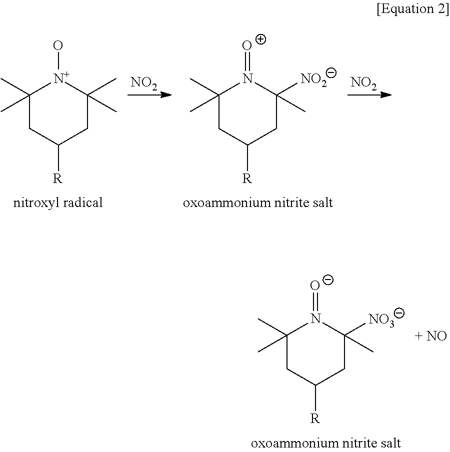

### Typical Nitroxide Radical—Oxoammonium Reactions

TEMPO is a stable nitroxide radical that can undergo a single-electron oxidation and produces a highly electrophilic oxoammonium species which serves as the active oxidizing agent in both covalent bond formation reactions and catalytic reactions. There are distinct differences between gas and liquid phase reactions. While the devices, methods and compositions described herein relate to a gas phase reaction, the following examples of liquid phase stable nitroxide radical—oxoammonium reactions provide the insight and guidance that led to the discovery of a way to regenerate TEMPO through variations in comproportionation/synproportionation mechanisms in covalent bond reactions and catalytic reactions in a gas/mist phase reaction, thereby dramatically extending the useful life of TEMPO in a gas polishing application of the type envisioned herein.

Equation 3 is a liquid phase reaction mechanism for stoichiometric oxoammonium reactions that involve comproportionation/synproportionation. This example describes the oxidation of an alcohol. The typical alcohol hydroxyl group (O—H) stoichiometry is replaced with the representation of nucleophilic site (O⁻) as an indication electronegativity of the oxygen in an aqueous solution. The first step in this reaction is identified with a “1”. The second step is identified as the comproportionation.

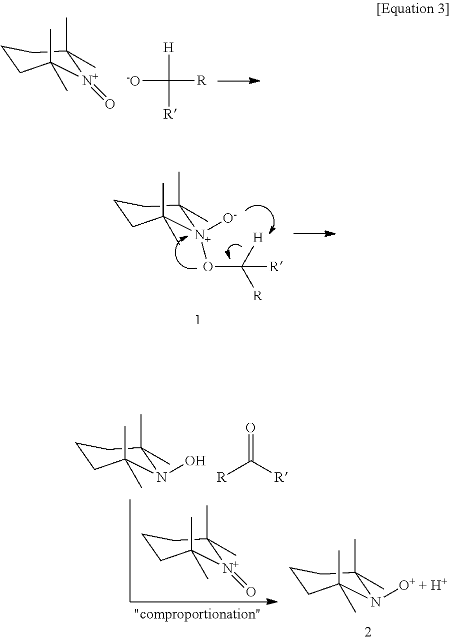

The comproportionation portion of this reaction shown in Equation 3 brings together two molecules containing the same elements: the hydroxyl amine reaction product from the first step of this overall reaction and another TEMPO molecule. The two molecules each contain the same elements but with different oxidation numbers. These form products in which both molecules have the same oxidation number. One of the reaction products of this second step is a newly created TEMPO molecule.

Equation 4 is a liquid phase reaction mechanism for nitroxide-catalyzed oxidations that involve N-oxoammonium intermediates as the active oxidizing agent. It was shown that details of the reaction mechanism depend on the compound used as the terminal oxidant in the reaction. For example, if a two-electron oxidant such as NaOCl is used as the terminal oxidant the nitroxide intermediaries are converted into oxoammonium molecules. One-electron terminal oxidants such as copper (II) operate via a more complex mechanism.

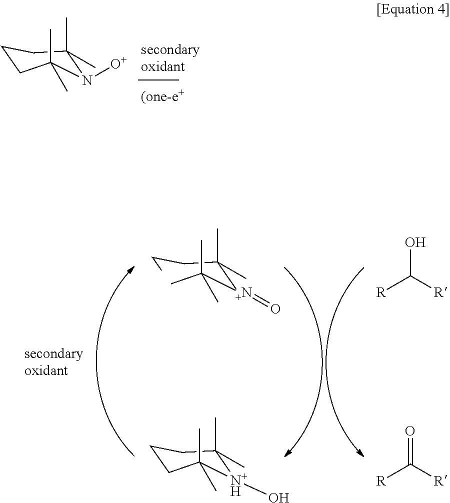

In some embodiments, the devices, methods and compositions described herein involve a combination of the liquid phase reactions shown above with the electrical bias described in the next section.

### NO₂ Containing Substrate

According to aspects of the presently disclosed embodiments, the formation of NO₂-sequestering/adsorptive/reactive surface comprises connecting TEMPO molecules, analogs thereof or derivatives thereof to a substrate material. According to other aspects of the presently disclosed embodiments, the NO₂-sequestering/adsorptive/reactive surface comprises TEMPO molecules, analogs thereof or derivatives thereof within a substrate material. In some embodiments, the NO₂-sequestering molecules can be dispersed within the substrate. In other embodiments, the NO₂-sequestering molecules can be dissolved within the substrate. In other embodiments, the NO₂-sequestering molecules can be attached through covalent bond to the surface of the substrate. The domain, or NO₂-sequestering region within a device may vary. In some embodiments, the entire inner surface of a device includes NO₂-sequestering material. In other embodiments, NO₂-sequestering domain is limited to the tubular structures. Control of the NO₂-sequestering particle size, domain size and its dispersion/solubility throughout the surface as well as the geometry provide multiple means to control the level of availability of NO₂-sequestering molecules to NO₂ molecules passing by. In some embodiments, the substrate material can be chosen from a variety of materials, including polymers or combinations of different types of polymers or homogenous material (e.g. carbon, glass) and others. In some embodiments, the substrates are chosen based on their physical properties (including, but not limited to, flexibility, porosity, durability, translucency, electrical conductivity, thermal conductivity, thermal stability, etc. or combination thereof) and their chemical properties (including, but not limited to, reactivity, solubility, stability, etc. or combination thereof).

TEMPO is provided as a non-limiting example, and it should be understood that there are many types of molecules (organic and inorganic) that can exhibit selective NO₂ sequestration. FIG. 5 and FIG. 6 show examples of organic molecules having selective NO₂ sequestration. Examples of inorganic molecules having selective NO₂ sequestration include, but are not limited to, soda lime, calcium hydroxide, potassium hydroxide, sodium hydroxide or combinations thereof.

In some embodiments, the NO₂ sequestration molecule(s) can comprise TEMPO, TEMPA, PROXYL, derivatives thereof and analogs thereof. In some embodiments, the compound can be TEMPA. TEMPA (2,2,6,6-tetra methyl-3,4-dehydro-piperidin-N-oxyl-4-acetylene) molecule is a variation on the 6-sided ring that is characteristic of the TEMPO family but has a double bond where the rest of the TEMPO group do not (see FIG. 5). In some embodiments, substitution on the TEMPO molecule can occur on the No. 4 carbon. In some embodiments, the compound can be a simple six-sided ring that includes one nitrogen (See FIG. 5). In some embodiments, PROXYL compounds that have 5 sided rings can be used (see FIG. 6). Some molecules in this group of five membered rings can have double bonds (e.g. 3-carbamoxyl-2,2,5,5-tetramrthyl-3-pyrrolin-1-yloxyl).

In some embodiments, the NO₂ sequestration molecule can be directly or indirectly attached or bonded to the substrate. In some embodiments, the TEMPO molecule comprises a substitution, also referred herein as a “handle” molecule or linker, that may in-turn interact with the substrate. In some embodiments, the handle attachment point to the TEMPO is covalently bonded to a carboxyl group that has been embedded on the carbon crystal structure. In some embodiments, the handle attachment point is the #4 carbon atom in the TEMPO molecule (4 substituted). In other embodiments, the handle attachment can substitute a hydrogen atom on the cycling ring of the TEMPO molecule.

The “handle” or linker creates a connection between the TEMPO and substrate. Handle molecule options include but are not limited to:

alcohol group

carboxyl group

amide group

hydroxide group

polar/non-polar molecules

Each type of handle has different characteristics, such as bond strength, photosensitivity, chemical stability, thermal stability, etc. Handles can be selected based on these properties as well as interaction with the TEMPO molecule or derivative thereof with a specific substrate. The substrate can be selectively bonded to by adjusting the reaction environment (for example, pH, temperature, presence/absence of a catalyst or combinations thereof) of the target bonding region.

In some embodiments, the TEMPO derivative can be TEMPOL which comprises a hydroxyl group (—OH) substitution on the Carbon No. 4.

Equation 5 is an example of TEMPOL forming a covalent bond to modified carbon.

In this reaction the COOH (carboxyl) group has been attached to activated carbon is reacting with the OH “handle” of the TEMPOL molecule to form product (3).

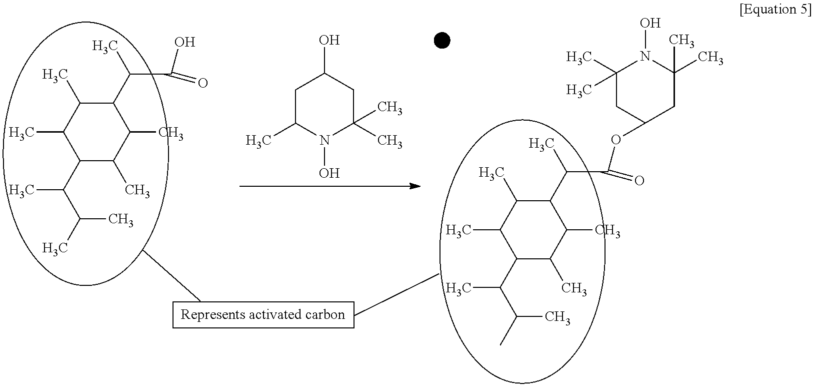

In the exemplary embodiment where carbon and TEMPO are covalently bonded, it is a COOH group that has been added to the surface of the carbon that reacts with the OH handle of the TEMPO. It should be appreciated that the example shows only one example of how we can combine TEMPO to polymers and monomers and crystalline materials like carbon.

In some embodiments, nitric acid is used to condition the substrate to enable substitution of TEMPO. In the first step of a two-step reaction, the nitric acid and carbon substrate are reacted to substitute OH groups and/or other handles onto the surface of the carbon-containing substrate. In the second step, activated carbon is reacted with TEMPO with an appropriate handle to facilitate attachment between the TEMPO molecule and substrate.

### Electrical Control of NO₂ Sequestration Process

According to some aspects of the presently disclosed embodiments, electrical bias voltage can be used to regulate the reversible formation of oxoammonium species derived from TEMPO or TEMPA. N-Oxoammonium salts in organic chemistry are a class of organic compounds sharing a functional group with the general structure R₁R₂N+=O X⁻ where X⁻ is the counterion. They are isoelectronic with carbonyls and structurally related to hydroxylamines and nitroxide radicals, with which they can interconvert via a series of redox steps. This feature has been demonstrated to provide a precise regulation of the rate of reaction between the oxoammonium species and the NO₂ in a gas phase reaction. It is also involved in the regeneration of TEMPO reactants.

In some embodiments, the composition or device comprising TEMPO, analog thereof or derivative thereof and the substrate composite can have electrochromic properties. In some embodiments, the color-changing property can be applied to a medical device for the indication of the state of the scrubbing material. For example, a color change can indicate the reactivity of the TEMPO. When a conducting polymer base material is exposed to a bias voltage, the TEMPO reactivity is influence and the state of that influence can be associated with a color change to the chromic material included in the substrate composite.

Equation 6 graphically explains the concept. The addition or removal of a DC bias voltage to an electrically conducting polymer with TEMPO, analog thereof or derivative thereof, covalently bonded to a monomer that is an integral part of the polymer, the following molecular changes to the TEMPO:

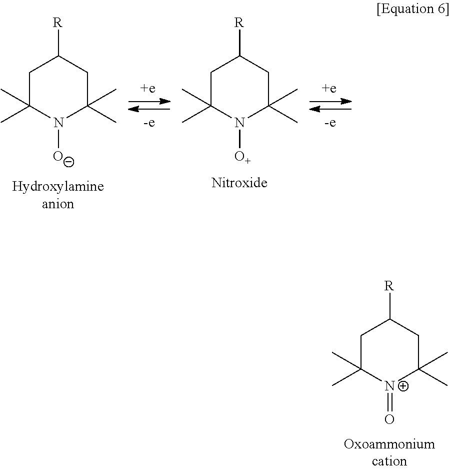

In some embodiments, the rate of reaction between a combination of a conducting monomer/polymer and covalently bonded TEMPO, analogs thereof or derivatives thereof or other members of the nitroxide radical group and available NO₂ are regulated by electrical charge/current control characteristics that are in a way similar to the effects caused by changes in a bias voltage applied to, and removed from, the gate of a “doped” silicon device like a transistor. The chemical characteristics affecting redox reactions of TEMPO and other members of the stable nitroxide radical group can be reversibly changed by the application of bias voltage to the molecule's “backbone” conducting polymer/substrate material.

In some embodiments, electrical bias voltage is applied in a single location. In other embodiments, electrical bias voltage is applied in multiple locations to the substrate material to regulate the effect across a surface. The thickness of the polymer coating/layer can be varied along the surface of a substrate to overcome inherent electrical resistance/impedance within the conducting base material. In some embodiments, the thickness of the polymer coating/layer is varied to make the electrical potential and thus the reactivity if the TEMPO molecules more uniform across a surface.

The change in applied voltage can dramatically and consistently influence the TEMPO/Oxoammonium reaction with NO₂ and other compounds as shown in Section 1 above. Therefore, it is expected that the observed ability to regulate oxoammonium reactions will apply to NO₂ molecules in a gas stream. This can be an attribute in the medical application envisioned for this process.

In some embodiments, reactivity of a TEMPO-containing surface on the lining of an inspiratory gas pathway can be modulated by varying the electrical voltage applied to the substrate material. This effect regulates the reaction by altering the affinity of TEMPO for NO₂. In some embodiments, the electrical potential applied to the surface is modulated in synchrony with pulsatile gas delivery so that TEMPO reactivity is maximized when gas concentrations are maximal. This approach can have the benefits of not using TEMPO attachment points when NO₂ levels are inherently low in the inspiratory gas, thereby prolonging the usable life of the TEMPO molecules, analogs thereof or derivatives thereof and extending the need to regenerate or reset the TEMPO molecules, analogs thereof or derivatives thereof. In other embodiments, the amount of NO₂ absorbed by a surface of TEMPO molecules, analogs thereof or derivatives thereof is regulated by the electrical charge applied to the substrate material.

In some embodiments, an electrical potential is applied to a TEMPO-containing surface to promote the release of NO₂ from the TEMPO molecules, analogs thereof or derivatives thereof. This approach can have the advantage of enabling a TEMPO-containing surface to be used, reset and reused in the process of NO₂ scrubbing. In some embodiments, an NO deliver device can reverse the flow of air within an inspiratory gas delivery lumen so that gas flow is away from the patient while simultaneously applying sufficient electrical potential for the TEMPO molecules to release NO₂. In some embodiments, NO₂ molecules released from the TEMPO molecules, analogs thereof or derivatives thereof, are directed through a carbon filter where they are sequestered. In other embodiments, NO₂ molecules are directed towards a source of vacuum. In other embodiments, NO₂ molecules are released into the atmosphere at a safe distance from the device user.

In some embodiments, an NO delivery system has a plurality of independent NO-delivery lumens, with some or all lined with TEMPO material for absorption of NO₂. In one embodiment, the system can simultaneously and independently deliver NO to the patient through one set of lumens while cleaning out the NO₂ from another set of lumens. In some embodiments, a NO delivery system can simultaneously apply an electrical potential to one or more specific lumens and not to another one or more specific lumens.

This ability to regulate the redox reactivity of nitroxide/oxoammonium reactions with other compounds appears to be directly applicable to regenerating the TEMPO as described herein. Preliminary research indicates that the TEMPO can be regenerated by introducing a mist containing a dual electron oxidant and a commensurate bias voltage. One example of a dual-electron oxidant is sodium hypochlorite. This has an added benefit of sterilizing a TEMPO-containing surface.

In use, a patient can be supplied with two NO₂-absorbing cannulas. While the patient uses one cannula, the other cannula can be reprocessed to remove NO₂ and/or sterilize. In some embodiments, byproducts from the dual electron oxidant process to regenerate the TEMPO are passed through an activated charcoal (carbon) filter for safe isolation. In some embodiments, the carbon filter is replaced at an appropriate frequency.

### Chromic Changes as Process Indicators

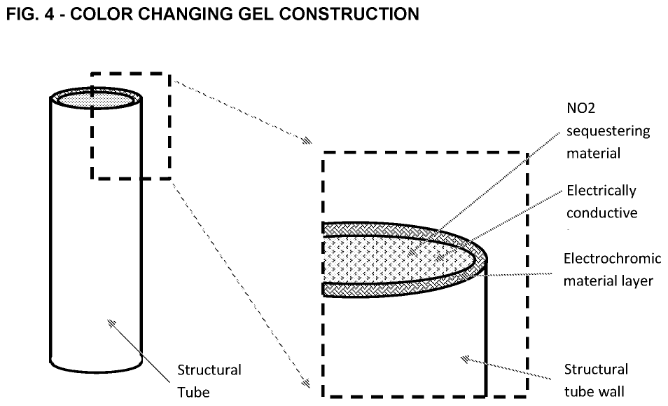

The substrate material that can include conducting polymer with NO₂ sequestering molecules (e.g. TEMPO) has two sides. One side is exposed to the NOx-laden gas stream, and the other is attached to a tube or other structure that provides structural support for the substrate material that may include conductive polymers. In some embodiments, a gel material that expresses chromic changes in response to the ionic changes in NO₂₋sequestering molecules (e.g. TEMPO) caused bias voltage change can be included in the substrate material. In some embodiments, the electrochromic material is a gel placed between the structural tube or other device and the conducting polymer (FIG. 4). In some embodiments, the electrochromic gel can respond with color change representative to the compounds used in the gel and applied voltage. Non limiting examples of electrochromic materials are: 1) tungsten trioxide, a material used in automatic tinting windows, 2) polyaniline polymers such as poly(2,5-dimethoxyaniline) (PDMA), 3) polythiophene polymers such as poly(3,4-ethylenedioxythiophene) (PEDOT) or poly(3-methylthiophene) (P3MT)), 4) Viologens (carbon-based salts such as 4,4′-bipyridine), 5) conjugated conducting polymers (thiophene, pyrrole, furan, carbazole, azulene, indole, aniline, polyethylenedioxythiophene (PEDOT)), 6) metal coordination complexes (2,2′-bipyridine, phthalocyanines, lutetium bis(phthalocyanine), 7) Prussian Blue (C₁₈Fe₇N₁₈), 8) metal cyanometallates, 9) polymer gel electrolyte containing perchlorate ions. In some embodiments, the required voltage for electrochromic polymers can be from 0 to 14 V DC. In some embodiments, electrical potential applied is in the 1.4 to 2.6V DC range.

In some embodiments, the purpose of the gel is to provide a color that can be used as an indicator of the electrical bias (also referred herein as electronic bias) associated with a conducting polymer. A change in polymer bias voltage can change the gel color. This is analogous to “mood rings” that change color with temperature. In some embodiments, gel color can be indicative that the bias voltage is present. This could be an indication of the battery charge status, external power status, or overall device status.

In some embodiments, bias voltage is used as a regulator for how reactive the TEMPO is with a gas stream. Equation 2 shows that nitrite salts have different reactivity than the nitroxide radical. Nitrate salts build up over time as TEMPO reacts with NO₂. The bias voltage can have the ability to influence the reactive surfaces on the TEMPO and therefore increase or decrease its ability to react with NO₂ in a way that is similar to but different than the reaction shown in equation 2. The voltage affects the number of available electrons and influences the TEMPO reactivity.

The color change can be valuable because it is indicative of the bias voltage value and the state of the TEMPO's reactivity with the NOx in a gas stream. In the case of an inspiratory air conduit, the bias voltage applied to the electrochromic lining can varied to indicate level of TEMPO reactivity based on the amount of NO₂ adsorbed. In other embodiments, the electrical charge causing the lining color change could be actively applied by the NO device as an indication of remaining life of the NO₂ adsorptive surface. In some embodiments, the monomer/polymers used as a point of attachment for TEMPO etc. provided herein are impervious to the gel and therefore the conducting monomer/polymer also acts as a barrier to prevent interaction between the gel and the TEMPO/oxoammonium molecules exposed to the gas stream with NOx (NO/NO₂).

### Resonance Imaging

Nitroxide radicals exhibit paramagnetism, due to the single electron spin of a free electron. This feature enables nitroxide radicals to be detected by common imaging techniques including electron paramagnetic resonance (EPR) spectroscopy and magnetic resonance imaging (MRI). As nitroxide radical-containing materials are incorporated into products, this ability to be imaged is retained. During manufacturing, imaging can be utilized as a quality step to inspect a product against requirements. For example, the presence/absence of a coating or thickness of a coating can be assessed in this way. For imaging purposes, nitroxide radicals can be present on the inner lumen of a tube or within the tube wall. In some embodiments, a catheter includes a layer of nitroxide radical material within its wall enabling visualization of catheter placement with MRI.

### Polymer Characteristics

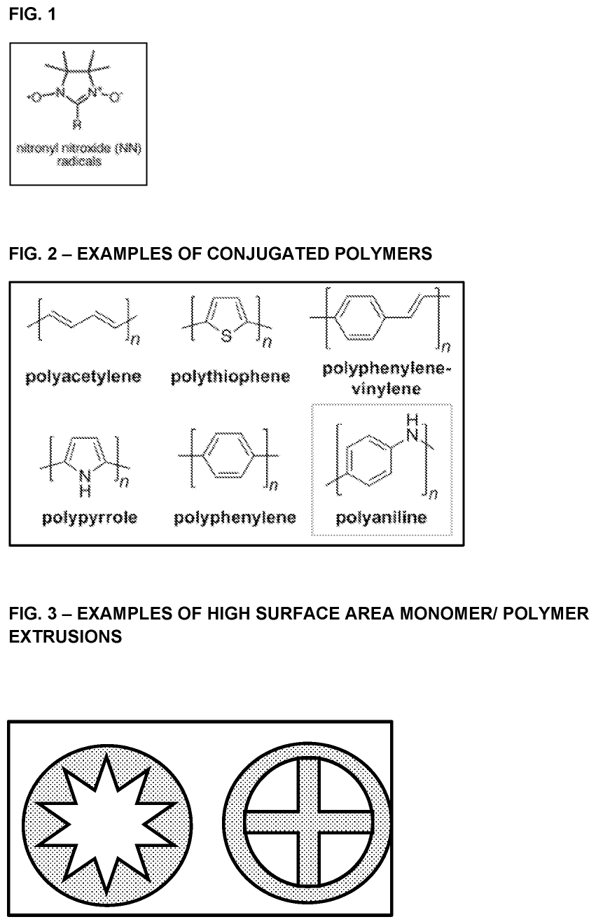

The bulk of commercial plastics are made up of various saturated organic polymers that are characteristically electrically classed as insulators. In contrast polymers that conduct electricity do so because they share a characteristic called molecular conjugation. A conjugated polymer has a system of connected p-orbitals across an intervening sigma bond and delocalized electrons interface by extending past the sigma bond. The pi electrons do not belong to a single bond or atom but rather to a group of atoms. Compounds with this characteristic can be cyclic, linear or mixed. FIG. 2 provides examples of conjugated polymers.

According to aspects of the present disclosure, the conjugated monomers/polymers used for NO₂ scrubbing combine the conductivity of traditional inorganic materials with many of the desirable properties of organic plastics including mechanical flexibility and low production costs. In some embodiments, the monomers/polymers can have an aromatic structure. Such monomers/polymers typically have excellent chemical, thermal, and oxidative stability. In many cases the monomers/polymers provided herein are practically insoluble in all common solvents due to the low hydrogen content. This solubility feature provides the required chemical isolation between the gel and the gas phase treatment on the other side of the conductive monomers/polymers

In some embodiments, Indium Tin Oxides (ITO) films can be applied to the gas-contacting surfaces of the NO conduits of an NO delivery device prior to TEMPO attachment.

In some embodiments, the conductive monomers/polymers do not comprise ITO. It should be appreciated that one of the most important advantages the use of a conductive monomers/polymers has over ITO material as the conductive medium is the method of fabrication. The conductive monomers/polymers used in this process are fabricated using ambient or moderate temperature aqueous chemical dispersion rather than the high vacuum and high temperature process required for the fabrication of ITO materials. The use of dispersion for production provides the opportunity to fine tune the characteristics of the substrate material in which the NO₂-sequestering molecule (e.g. TEMPO) is contained.

In some embodiments, the TEMPO substrate material is an electrically conductive polymer with discrete domains for a NO₂-sequestering molecule. In some embodiments, these domains consist of pores in the substrate material. Control of the particle size and domain size and its dispersion throughout the surface as well as the geometry provide multiple means to control the level of availability of NO₂-sequestering molecules to NO₂ molecules passing by. This control is typically done as part of the design and manufacturing processes. In some embodiments, however, active control of domain size can be done through electrical properties of the materials to vary the pore size and affect the level of interaction between NO₂ containing gas and NO₂ sequestering molecules.

It should be appreciated that the pore size selection can be tailored to the size of the molecules that will be interacting with the NO₂-sequestering molecules, such as TEMPO, TEMPA, PROXYL compounds, analogs thereof, derivatives thereof or combinations thereof. In some embodiments, the pore size can be big enough to allow easy access but not much larger than “accommodating” because excessive pore size reduces surface area. The charge or partial charge on molecules can also play a role in pore size. For example, a highly porous surface coated with NO₂-sequestering molecules (e.g. TEMPO molecules) could contain a majority of NO₂-sequestering molecules deep within the pores of the substrate molecular surface, thereby limiting the NO₂-adsorption capabilities of the surface. Contrastingly, a surface with small pore size would have less surface area overall, but more TEMPO molecules in contact with the NO₂-containing gas. Aspects such as surface area, particle size, particle count, lumen size, pore size, pore count and cannula construction are interdependent variables that can lead to multiple functional permutations by one skilled in the art. Furthermore, mixing and gas flow state (turbulence vs. laminar) contribute to the level of interaction between NO₂-containing gas and NO₂-sequestering materials. In some embodiments, the surface finish within a gas flow lumen can be intentionally rough to disturb the gas flow and promote turbulence and mixing. In some embodiments, the lumens are textured. In some embodiments, mixing elements can be included within the gas flow path to promote mixing and turbulence.

Substrate surfaces can have a variety of topologies. This can affect the efficacy of a NO₂ scrubber if NO₂-sequestering molecules (e.g. TEMPO, TEMPA or PROXYL molecules) are located at the bottom of pores/valleys where there is less exposure to gas to react with. In the carbon substrate example, by changing the reaction environment with the nitric acid, the surface of the carbon can be tailored and shaped to obtain a desired pore size, analogous to etching of the surface. By choosing the optimal pore size and distribution, NO₂₋sequestering molecules will be located at the surface, rather than at the bottom of the pores for increased NO₂ scavenging. In some embodiments, the average pore size can be about 30 nm. In some embodiments, pore size can be from 20 to 40 nm, from 30 to 35 nm, from 20 to 35 nm, from 30 to 40 nm, from 25 nm to 30 nm etc. . . . .

Optimal adsorption can be determined empirically by varying temperature, pH and exposure time of the nitric acid and carbon to vary the pore sizes. Experimentally, one can determine which combination yields the highest NO₂-sequestration/adsorbtion/reaction.

The temperature of the reaction can change the location of the reaction with respect to the pores. In some embodiments, the substrate surface can be conditioned by using a specific reaction environment, and thereby tailor the points on the carbon substrate that reactions will occur.

In some embodiments, the substrate material can include carbon in a pure form such as graphite, nanotubes, Bucky balls and the like. Nanotubes and Bucky balls provide a more flexible substrate.

The conductive polymers do not have the high structural capabilities seen in many non-conducting polymers. This limitation requires that conductive monomers/polymers be adhered to another polymer with increased mechanical properties. In some embodiments, conductive polymers and other components of the substrate material are made into a sleeve that is protected by a structural tube. In other embodiments, conductive polymers and other components of the substrate material are deposited as a coating on the lining of a structural tube that conveys an inspiratory gas to a patient. NO₂-sequestering molecules (e.g. TEMPO molecules etc.) are attached or integrated into the substrate material in process specific sequences. In one embodiment, a polymer material is compounded with a NO₂-sequestering material and extruded into a tube. In another embodiment, a NO₂-sequestering material is compounded with another material, applied as a coating on a surface and activated with a secondary reaction (for example acid flush). In another embodiment, the process comprises the steps of: flushing a tube with a primer material to increase surface energy, flushing with a liquid including NO₂-sequestering material, and curing to adhere, stabilize and/or activate the inner lining of the tube. Curing steps can include UV light exposure, heat exposure, time, catalyst flush, a pH-specific flush or any combinations thereof.

When a bias voltage is applied to the conducting polymer portion of the substrate material, the orientation of surface electrons of the conductive layer of the substrate material is changed which in-turn affect the reactivity of the TEMPO molecule. As an example, one of the conductive monomers applicable to this process is 4-carboxy-N,N-diphenylaniline-2,2,6,6-tetramethylpiperidin-1-yloxy (TPAT). The synthesis of TPAT monomer is summarized in Equation 7.

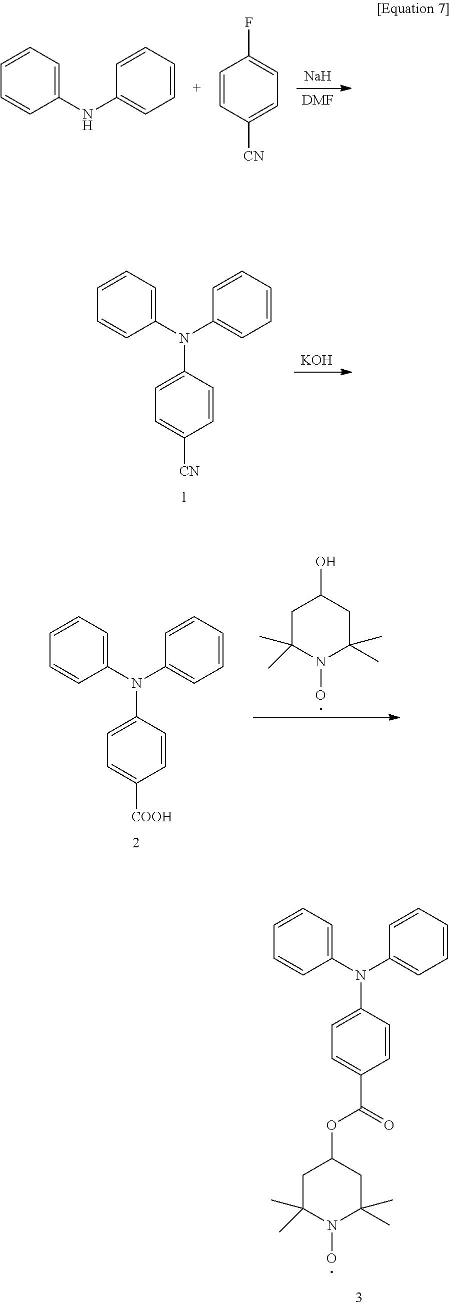

### Synthesis of the TPAT Monomer:

Synthesis of compound 1 (4-cyano-N,N-diphenylaniline) Diphenylamine.

1. (5.1 g), sodium hydride (1.5 g) and N,N-dimethylformamide (DMF, 50 mL) is firstly mixed in a pre-dried flask, and then 4-fluorobenzonitrile (4.5 g) is added.

The mixing is performed under a N₂ atmosphere for 12 h at 110° C.

After cooling, the resulting solution is extracted with CH₂Cl₂ and dried using anhydrous MgSO₄. The 4-cyano-N,N-diphenylaniline (1) is isolated by column chromatography. The expected result is 4.98 g as a yellow powder.

2. Synthesis of compound 2 (4-carboxy-N,N-diphenylaniline) 4-Cyano-N,N-diphenylaniline. (1.0 g) and KOH (2.1 g) is firstly dissolved in a mixture of deionized water (30 mL) and glacial acetic acid (20 mL) in a pre-dried three-necked flask and heated at reflux (85° C.) for 48 h.

After cooling, hydrochloric acid (1 M) is added dropwise to the reaction solution until the pH value of the reaction solution was adjusted to about 1. As the pH changes a white precipitate will appear this is isolated by filtration and washing with a large amount of deionized water.

The obtained white powder of 4-carboxy-N,N-diphenylaniline is dried in vacuum at 60° C. for 24 h. The expected yield is 0.82 g.

3. Synthesis of compound 3 (4-carboxy-N,N-diphenylaniline-2,2,6,6-tetramethylpiperidin-1-yloxy). Combine 1.6 g of 4-Hydroxy-2,2,6,6-tetramethylpiperidine 1-oxyl free radical and 2.0 g of 4-carboxy-N,N-diphenylaniline. These compounds are dissolved in 50 ml of CH₂Cl₂ in a pre-dried three-necked flask, then 0.4 g of 1-(3dimethylaminopropyl)-3-ethylcarbodiimide hydrochloride as the dehydrating agent and 1.6 g of 4-dimethylaminopyridine as the acylating catalyst are added, and the mixture is stirred for 24 h at room temperature. Ideally the mix should have a N₂ gas blanket while mixing.

4. The reaction mixture is then separated by vacuum filtration. The filtrate is washed with saturated brine three times and the organic phase is dried using anhydrous Na₂SO₄. The ester is purified using column chromatography with silica gel and petroleum ether/ethyl acetate. The yield is 1.9 g of a pink powder.

### Mass Transfer

It should be appreciated that the effectiveness of a gas phase/solid phase reaction depends upon the accessibility of the gas to the active sites on the solid phase.

In general, polymers can possess plentiful conformational and rotational flexibilities which enable them to maximize intermolecular and intramolecular cohesive interactions and packing space efficiently in the solid state. Consequently, the free volume within a polymer, which is the space within the material not occupied by the polymer molecules, can vary remarkably due to certain factors (physical state, molecular structure, etc.). Hence, if the material selected for NO₂ scrubbing owns a sufficiently large free volume, interconnectivity will occur, and nano-porosity will be significantly presented.

This understanding led to the development of conducting polymers that expose the active TEMPO/oxoammonium molecules to gas on a structure with high nano-porosity. Research into the various methods used to create nano-porosity led to the use of supercritical carbon dioxide (scCO₂). scCO₂ is the most common substance employed, which, once turned into the gas phase, acts as a porogen to generate pores within the polymer or monomer. The scCO₂ is ideal and clean agent because CO₂ is chemically stable and nontoxic. In addition, it has a mid-critical temperature of 31° C. and relatively high solubility in most monomer/polymer solutions.

The shape of the monomer/polymer also contributes to the surface area. FIG. 3 provides two examples of possible extrusion configurations. These structures provide high internal surface area for increased TEMPO-gas contact as gas travels through the extrusion.

### Medical Device Applications

In some embodiments, nitric oxide delivery to a patient involves either sourcing NO from a reservoir or generating NO on site. NO converts to NO₂ over time. NO₂ can be removed by a selective NO₂-reactive material within one or more lumens of a tubular structure that carries a gas containing NO and/or NO₂.

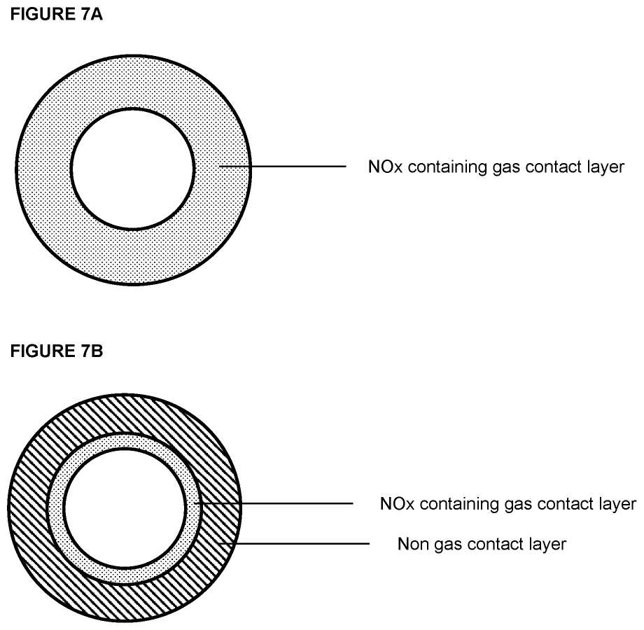

FIG. 7A and FIG. 7B depicts a NO₂-sequestering gas contact layer and how it is located within a tubular structure. In some embodiments, the NO₂-sequestering material is a component of the entire tubular structure. In other embodiments, the NO₂-sequestering material is a layer that covers at least a portion of the inner surfaces of a more structural tube that conveys the NO. In some embodiments, the NO₂-sequestering material is a layer that covers the entirety of the tubular structure. In some embodiments, a NO₂-sequestering layer is a component of a tube-like structure that is inserted into another structural tubing, like a lining or sleeve. In other embodiments, the NO₂-sequestering layer is a coating. Control of the particle/domain size and its dispersion throughout the surface as well as the geometry provides multiple means to control the level of availability of NO₂ sequestering molecules to NO₂ molecules passing by. The general configuration is designed to provide sufficient residence time and exposure to the NO₂-sequestering molecule to reduce the concentration of NO₂ to desired concentrations for the desired “lifetime” of the device. Desired concentrations are typically clinically accepted concentrations, ranging from zero to about 20 ppm, depending on the length of exposure. In some embodiments, the configuration includes tubular structures, manifold, chamber, mixing element or other gas-containing structures.

In some embodiments, a tube and/or tube lining is constructed from a material that has been compounded with TEMPO and/or derivatives or other NO₂-sequestering molecules. TEMPO can be compounded with materials including, but not limited to, polyethylene, polypropylene, polyamide, styrene-butadiene-styrene, thermoplastic elastomer, polyurethane, polyvinylchloride, and combinations thereof.

In some embodiments, an NO₂-sequestering material is dispersed/dissolved in a coating. In some embodiments, the coating includes polyamide, styrene-butadiene-styrene, thermoplastic elastomer, polyurethane, polyvinylchloride, or a combination thereof. In some embodiments, the gas-contacting surfaces of a tube are coated. In other embodiments, a an open-cell structure (like a foam) that provides high surface area is coated. In some embodiments, the open-cell structure is entirely comprised of NO₂-sequestering material or a compound of NO₂-sequestering material and one or more other materials.

In some embodiments, TEMPO alone and/or with derivatives or other NO₂-sequestering molecules with varying handles can also be used in a pellet form in a scrubber cartridge. In some embodiments, the handle connected to the TEMPO, derivatives thereof or other NO₂-sequestering molecules provides a functional benefit, such as desiccation. In some embodiments, the handle provides orientation of the TEMPO molecules derivatives thereof or other NO₂-sequestering molecules. As an example of orientation, in some embodiments, the handle molecule creates a crystal at the center of a sphere such that NO₂-sequestering molecules (e.g. TEMPO molecules) on the periphery of the crystal are oriented with the active, NO₂-sequestering end facing outward.

In some embodiments, ionic bonds are used to form the center of a structure with NO₂-sequestering molecules (e.g. TEMPO molecules) around the periphery. This approach could facilitate the process of transferring the NO₂-sequestering molecules in pellet for coating a surface in a process. In some embodiments, water can be used as a solvent of NO₂-sequestering molecules crystals (e.g. TEMPO crystals).

In some embodiments, the device for delivering nitric oxide (NO) comprises a nasal cannula comprising an intra-lumen, the intra-lumen having a surface comprising a NO₂-sequestering molecule, and wherein the intra-lumen surface is configured to contact the gas stream; an inlet to deliver a gas stream comprising NO and NO₂; and an outlet configured to deliver NO enriched gas. In some embodiments, the NO₂-sequestering molecule comprises TEMPO, TEMPA, analogs thereof, derivatives thereof or combinations thereof. In some embodiments, the surface comprises an electrically conductive polymer. In some embodiments, the NO₂-sequestering molecule is embedded into the electrically conductive polymer. In some embodiments, the NO₂-sequestering molecule is attached to the electrically conductive monomer that is part of a polymer. In some embodiments, the electrically conductive polymer is configured to alter the NO₂-sequestering molecules (e.g. TEMPO molecules, analogs thereof, derivatives thereof or combinations thereof) upon application of an electrical charge to the NO₂ sequestering molecules. In some embodiments, the NO₂-sequestering molecules (e.g. TEMPO molecules, analogs thereof, derivatives thereof or combinations thereof) can be chemically attached to the electrically conductive monomer that is part of a polymer through a linker. In some embodiments, the surface comprising the NO₂-sequestering molecules (e.g. TEMPO molecules, TEMPO derivatives, TEMPO analogs or combinations thereof) are configured to be reset upon application of an electrical potential to the surface so that NO₂ is released from the surface. In some embodiments, the NO₂-sequestering molecules (e.g. TEMPO molecules, TEMPO derivatives, TEMPO analogs or combinations thereof) are covalently bonded to electrically-conductive monomer that is part of a polymer so that changes in electrical potential applied to the monomer/polymer increase or decrease the NO₂-sequestering molecules affinity for NO₂. In some embodiments, the device further comprises a coating on one or more inspiratory-gas-contacting surfaces within the nasal cannula. In some embodiments, the device further comprises an electrochromic gel configured to change color based on an electrical voltage. In some embodiments, the color change of the electrochromic gel is indicative of the propensity of the NO₂-sequestering molecules to react with NO₂.

In some embodiments, a filter is located between the NO₂ scrubbing portion of a NO delivery device and the patient to capture particulates. In some embodiments, the filter has a pore size ranging from 0.1 to 12 microns. In some embodiments, the filter has a pore size ranging from 0.1 to 0.5, 0.1. to 1, 0.1 to 2, 0.1 to 3, 0.1 to 4, 0.1 to 5, 0.1 to 6, 0.1 to 7, 0.1 to 8, 0.1 to 9, 0.1 to 10, 0.1 to 11, 0.1 to 12 microns. In some embodiments, the filter has a pore size ranging from 0.1 to 0.2, 0.1. 0.1 to 0.3, 0.1 to 0.4, 0.1 to 0.5, 0.1 to 0.6, 0.1 to 0.7, 0.1 to 0.8, 0.1 to 0.9, 0.1 to 1 micron. In some embodiments, the filter has a pore size of 0.22 micron. Filter pore sizes can vary however, with typical pore sizes from 0.1 to 20 micron. Coatings that include NO₂ sequestering materials can be used on fibrous, non-woven, sintered, and cellular materials for NO₂ sequestration as well as particulate filtration. In other embodiments, the filter is constructed of a material that has been compounded with NO₂ sequestering materials.

In some embodiments, NO₂-sequestering molecules (e.g. TEMPO molecules, TEMPO derivatives, TEMPO analogs or combinations thereof) can be used to coat fibrous, non-woven, sintered, and cellular materials for NO₂ absorption as well as particulate filtration.

Aspects of the disclosure provide devices comprising a coating on one or more inspiratory-gas-contacting surfaces within the nasal cannula or other device that exposes the gas to the NO₂-sequestering molecules. Other inspiratory-gas-contacting devices include, but are not limited to, ventilator tubing, respiratory humidifier components, respiratory particulate filters, endo-tracheal tubes, tubing connectors, nasal prongs, respiratory masks, HEPA filters, piping within a ventilator, piping within a CPAP machine, piping within an Oxygen concentrator, piping within an anesthesia machine, inhalers, blood-gas exchange systems (e.g. ECMO devices), wound healing devices, HVAC systems, devices for smoking, and other gas handling components and devices. In some embodiments, the device further comprises an electrochromic gel configured to change color based on an electrical voltage. In some embodiments, the color change of the electrochromic gel is indicative of the propensity of the TEMPO molecules, TEMPO derivatives, TEMPO analogs, or combinations thereof to bind with or remove NO₂.

In some embodiments, the devices, methods and compositions provided herein can also be used in NO gas-handling components for wound healing and blood-gas exchange (e.g. Extracorporeal membrane oxygenation (ECMO)).

In some embodiments, the devices, methods and compositions provided herein relate to nitric oxide (NO) delivery for use in various applications, for example, inside a hospital room, in an emergency room, in a doctor's office, in a clinic, and outside a hospital setting as a portable or ambulatory device. An NO generation and/or delivery system can take many forms, including but not limited to a device configured to work with an existing medical device that utilizes a product gas, a stand-alone (ambulatory) device, a module that can be integrated with an existing medical device, one or more types of cartridges that can perform various functions of the NO system, and an electronic NO tank. The NO generation system uses a reactant gas, including but not limited to ambient air, to produce a product gas that is enriched with

NO.

An NO generation device can be used with any device that can utilize NO, including but not limited to a ventilator, an anesthesia device, a defibrillator, a ventricular assist device (VAD), a Continuous Positive Airway Pressure (CPAP) machine, a Bilevel Positive Airway Pressure (BiPAP) machine, a non-invasive positive pressure ventilator (NIPPV), a nasal cannula application, a nebulizer, an extracorporeal membrane oxygenation (ECMO), a bypass system, an automated CPR system, an oxygen delivery system, an oxygen concentrator, an oxygen generation system, and an automated external defibrillator AED, MRI, and a patient monitor. In addition, the destination for nitric oxide produced can be any type of delivery device associated with any medical device, including but not limited to a nasal cannula, a manual ventilation device, a face mask, inhaler, or any other delivery circuit. The NO generation capabilities can be integrated into any of these devices, or the devices can be used with an NO generation device as described herein.

While the foregoing written description enables one of ordinary skill to make and use what is considered presently to be the best mode thereof, those of ordinary skill will understand and appreciate the existence of variations, combinations and equivalents of the specific embodiments, method, and examples herein. The presently disclosed embodiments should therefore not be limited by the above described embodiment, method and examples, but by all embodiments and methods within the scope and spirit of the presently disclosed embodiments.

1. A device for delivering a gas containing nitric oxide (NO) and/or nitrogen dioxide (NO₂) comprising:
one or more inlets;
one or more lumens comprising one or more surfaces configured to be in contact with the NO/NO₂ containing gas stream, wherein the one or more gas contact surfaces comprise a nitrogen dioxide sequestering (NO₂-sequestering) molecule; and
one or more outlets.

2. (canceled)

3. (canceled)

4. (canceled)

5. (canceled)

6. (canceled)

7. (canceled)

8. (canceled)

9. (canceled)

10. (canceled)

11. (canceled)

12. The device of claim 1 further comprising
an electrically conductive material configured to be in contact with the nitrogen dioxide NO₂-sequestering molecule.

13. The device of claim 1, wherein the device is a nasal cannula.

14. The device of claim 1, wherein the one or more lumens are a component of a multilayer and/or multilumen structure.

15. The device of claim 1, wherein the one or more lumens are textured.

16. The device of claim 1, wherein the NO₂-sequestering molecule is a molecule that sequesters NO₂.

17. The device of claim 1, wherein the nitrogen dioxide sequestering molecule includes TEMPO, 4-substituted TEMPO derivatives, PROXYL and derivatives, TEMPA and derivative, calcium hydroxide, sodium hydroxide or combinations thereof.

18. The device of claim 1, wherein the one or more gas contact surfaces contain an indicator indicative of the propensity of the NO₂-sequestering to sequester NO₂.

19. The device of claim 1, wherein the NO₂-sequestering molecule can be reset.

20. The device of claim 1, wherein the one or more gas contact surfaces comprise the NO₂-sequestering molecules dissolved therewithin, dispersed therewithin or chemically bound thereto.

21. (canceled)

22. (canceled)

23. A device for delivering nitric oxide (NO), the device comprising:
one or more lumens, the one or more lumen having one or more surfaces containing a nitrogen dioxide (NO₂) sequestering molecule, wherein the one or more surfaces are configured to contact a gas stream;
one or more inlets configured to receive the gas stream comprising NO, NO₂ or NO and NO₂; and
one or more outlets configured to deliver NO enriched gas.

24. The device of claim 23, wherein the NO₂ sequestering molecule includes one or more nitroxide radical containing compounds.

25. The device of claim 23, wherein the NO₂-sequestering molecule includes TEMPO, TEMPA, TEMPO derivatives, TEMPO analogs or combinations thereof.

26. The device of claim 23, wherein the NO₂-sequestering molecule is a molecule that sequesters NO₂.

27. The device of claim 23, wherein the NO₂-sequestering molecule selectively reacts with NO₂.

28. The device of claim 23, wherein the NO₂-sequestering molecule selectively binds with NO₂.

29. The device of claim 23, wherein the one or more surfaces comprise an electrically conductive polymer.

30. (canceled)

31. (canceled)

32. The device of claim 29, wherein the NO₂-sequestering molecule is embedded in an electrically conductive monomer/polymer, wherein the electrically conductive monomer/polymer conveys variations in electrical bias to the NO₂-sequestering molecule, thereby regulating the chemical reactivity of the NO₂-sequestering molecule.

33. The device of claim 29, wherein the NO₂-sequestering molecule is chemically attached directly or through a linker molecule to an electrically conductive monomer that is part of the electrically conductive polymer.

34. (canceled)

35. (canceled)

36. The device of claim 29, wherein the NO₂-sequestering molecule includes TEMPO, TEMPA, TEMPO derivatives, TEMPO analogs, or combinations thereof, and wherein the TEMPO, TEMPA, TEMPO derivatives, TEMPO analogs, or combinations thereof, are bonded to electrically-conductive monomer that is part of the polymer so that changes in electrical potential applied to the monomer/polymer increase or decrease the affinity of the TEMPO, TEMPA, TEMPO derivatives, TEMPO analogs, or combinations thereof, for NO₂.

37. (canceled)

38. (canceled)

39. The device of claim 23, wherein the device is a nasal cannula and further comprises a coating on one or more inspiratory-gas-contacting surfaces within the nasal cannula.

40. (canceled)

41. The device of claim 29, further comprising an electrochromic gel adjacent or integrated into the conductive polymer and configured to change color based on changes in electrical bias/electrical voltage applied to the conductive polymer.

42. (canceled)

43. A method for enriching a gas stream in nitric oxide, the method comprising:
delivering a gas stream comprising nitric oxide (NO), nitrogen dioxide (NO₂) or NO and NO₂ onto a substrate comprising a NO₂-sequestering molecule, such that the gas stream contacts the NO₂-sequestering molecule; and
enriching the gas stream with NO by the removing NO₂ from the gas stream thereby generating a NO enriched gas stream.

44. (canceled)

45. (canceled)

46. (canceled)

47. (canceled)

48. (canceled)

49. (canceled)

50. (canceled)

51. (canceled)

52. (canceled)

53. (canceled)

54. (canceled)
